(19) United States
(12) Patent Application Publication
(10) Pub. No.: US 2013/0034524 A1
(43) Pub. Date: Feb. 7, 2013

(54) Non-Enzymatic Method for Harvesting Adipose-Derived Stromal Cells and Adipose-Derived Stem Cells from Fat and Lipo-Aspirate

(71) Applicant: Siamak Agha-Mohammadi, Newport Coast, CA (US)

(72) Inventor: Siamak Agha-Mohammadi, Newport Coast, CA (US)

(21) Appl. No.: 13/197,393

(22) Filed: Aug. 3, 2011

(51) Int. Cl. C12N 5/0775; A61K 35/12

(52) U.S. Cl. 424/93.7; 435/366

(57) ABSTRACT

The present disclosure relates generally to processes, devices and systems for separating and concentrating stem and stromal cells from adipose tissue using a combination of mechanical disruption and filtration-centrifugation to obtain a highly enriched population of stem cells.

### BACKGROUND OF THE INVENTION

1. Field of the Invention

The present disclosure relates generally to processes, devices and systems for separating and concentrating stem and stromal cells, also known as regenerative cells, from adipose tissue, more specifically to a defined process of extracting, separating and concentrating clinically useful regenerative cells from adipose tissue using a combination of mechanical disruption and filtration-centrifugation to obtain a highly enriched population of stem cells.

2. Background Information

Regenerative medicine is a rapidly growing field that uses laboratory-grown or therapeutically-induced human tissue as a replacement for treating injuries, diseases, or cosmetic applications. As such, there has been astounding new advancements in the ability to repair or replace damaged human tissue with the use of stem cell related treatments and technology. Before, remedies for damaged tissue or organ functions due to deformities, injuries, diseases or simple wear-and-tear relied upon either the body's ability to repair itself (or not), or a surgeon's skilled hands. Now, the area of regenerative medicine promises to revolutionize our ability to remediate countless physical and mental disorders, maladies and ailments that have perpetually afflicted humans since the dawn of time. Where once there was no solution or remedy, researchers and doctors are now at the brink of overcoming such limitations by developing the technology to re-grow and stimulate damaged tissue and restore organ functions through the use of tissue engineering and stem cell therapy.

One such aspect of regenerative medicine revolves around taking advantage of the capability of regenerative cells found within adipose tissues. These stem cells and progenitor cells have the ability to renew themselves indefinitely and develop into mature specialized cells. It is known that stem cells are found within embryos during early stages of development, in fetal tissue and in most adult organs and tissue. Embryonic stem cells (hereinafter referred to as “ESCs”) are known to become many if not all of the cell and tissue types within the human body. ESCs not only contain all the genetic information of the individual (like most cells) but also contain the inherent capacity to become any of the 200+ cells and tissues of the body. Thus, it is believed that these cells have tremendous potential for regenerative medicine as a whole. For example, in studies it has been shown that ESCs can be grown into specific tissues such as heart, lung or kidney which may then be used to repair damaged and diseased organs. But so far, ESC derived tissues have shown their restriction in a clinical setting. Because ESCs are derived from another individual, i.e., an embryo, there is a risk that the recipient's immune system may reject the new biological material. Although immunosuppressive drugs are available that suppress such rejection, “anti-bodies” are known to block necessary immune responses such as those against bacterial infections and viruses. Additionally, there is the vast ethical debate over the source of ESCs, i.e., embryos, exposes an additional and, perhaps, challenging obstacle in the foreseeable future.

Adult stem cells (hereafter noted as “ASCs”) represent an alternative to the use of ESCs. For many years unbeknownst to science ASCs have resided quietly in many adult organs and tissues, presumably waiting to respond to trauma or infection, or disease processes so that they can regenerate the injured tissue. There has been developing scientific evidence that indicates each human (mammal) individual carries a pool of ASCs that contain many of the abilities of the precursors, ESCs, to become many if not all types of cells and tissues within the human body. Therefore, ASCs, like ESCs, have remarkable potential for use in a clinical setting and in regenerative medicine.

ASC populations have been shown to be present in many tissue types including bone marrow, skin, muscle, liver and brain. However, the frequency of ASCs in these tissues is low. For example, general mesenchymal stem cell populations in bone marrow is estimated at between 1 in 100,000 and 1 in 1,000,000 nucleated cells. Correspondingly, extraction of ASCs from skin unfortunately involves a complicated series of cell culture processes over several weeks and clinical application of skeletal muscle-derived ASCs requires a two to three week culture phase. Thus, any proposed clinical application of ASCs from such tissues requires culture and proliferation, purifying, and maturing by processes of cell isolation and cell culture.

While cell culture of ASC's may provide a way of increasing numbers, purity, and maturity, they unfortunately do so at a steep cost clinically, logistically and fiscally. This can include one or more of the following technical difficulties: loss of cell function due to cell maturation, loss of potentially useful non-stem (stromal) cell populations, delays in potential application of regenerative cells to patients, increased monetary budget, and increased risk of contamination within the microenvironment during culture. Recent studies investigate the therapeutic effects of bone-marrow derived ASCs have used essentially whole marrow to circumvent the problems associated with cell culturing. The clinical benefits, however, have been insufficient and can derive the outcome almost certainly being related to the limited ASC population (dose) available within bone marrow tissue.

During the past 10 years, studies have shown that adipose tissue can be an excellent source of ASCs. Unlike marrow and skin, adipose tissue is relatively simple to harvest in great quantities. Further, studies have demonstrated that adipose derived ASCs possess pluripotency, or the ability to go down several cell line lineages, in vitro, that include connective tissue (bone, muscle, cartilage), as well as cardiac and nerve tissue. This clearly demonstrates that adipose tissue presents an ideal source for ASCs for use in regenerative medicine.

However, there are not many suitable methods for harvesting adipose derived ASCs in today's medical practices. Existing methods contain a number of deficiencies that may include the ability to optimally accommodate an aspiration (liposuction) device for proper removal of adipose tissue. For example, many methods of lipoaspirating the tissue involve methods that can damage and harm the cells (laser, water and ultrasound) which are sometimes largely ignored. Other approaches may also contain a partial or full automation process of harvesting of adipose tissue which possesses limits due to poor engineering, the limits of their methods or cost to produce. Additionally, this equipment lacks the sophistication to take the tissue through the appropriate steps to maintain viability and purity of the sample. Further, systems may lack volume capacity as well. There are also existing methods that are deficient in being only a partially closed system from harvesting (lipo-aspiration) into the processing and reinsertion of the concentrated tissue. Further, adipocyte derived stem cells are typically isolated using enzymatic methods, and depending on the type of dissociating agent used, may cause stem cells so isolated to differentiate. Moreover, studies have shown that consistent isolation of ADSCs is particularly dependent on the protease formulation, including that such methods suffer from inconsistencies with respect to nucleated cells, viability and frequency of specific lineages.

Finally, existing systems lack affordable components for disposability or lack the ability to be decontaminated via autoclave, which attenuates the risks of cross-contamination of material from one sample to another. In summary, the many prior techniques and systems for harvesting ASCs from adipose tissue do not initially appear to overcome the technical and safety complications associated with other methods of harvesting ASCs from other tissues. This clearly demonstrates the need for processes, systems and methods that are competent, safe and affordable in harvesting and reinserting regenerative cell populations from adipose tissue that will increase yield, maintain purity or the ability to purify, and be capable of effectively, reliably and safely perform these necessary functions.

Ideally, such a device, system or method will yield regenerative cells in a manner suitable for direct placement into a recipient. To this purpose, the system or method of the present disclosure is optimized such that direct placement or reinsertion of the regenerative cells from the system into the patient does not incite any adverse effects within the patient that may include the presence of unsafe levels of enzymes, toxins, infectious agents, bacteria, and other potentially harmful additives.

### SUMMARY OF THE INVENTION

The present disclosure relates to processes, devices and systems for separating and concentrating stem and stromal cells, specifically from adipose tissues, using a combination of mechanical disruption and filtration-centrifugation in the absence of enzymatic dissociating agents to obtain a highly enriched population of stem cells.

In embodiments, a method of enriching a stem or stromal cell population from adipose tissue in the absence of a dissociating reagent is disclosed including injecting a physiological infiltration fluid (PIF) into a subject in one or more areas where fat is to be removed, mechanically dissociating fatty tissue in the infiltrated area with a tubular device and removing the dissociated fatty tissue and PIF via aspiration; collecting a first lipo-aspirate in a first vessel in fluid communication with the tubular device, where the first vessel comprises one or more first filters, and where the filters are configured to separate fatty tissue from fluid that contains the mesenchymal cell population resulting in a second lipo-aspirate; and concentrating the mesenchymal cell population of the second lipo-aspirate (via centrifugation or) in a second vessel in fluid communication with the first vessel via filter-centrifugation, where one or more filtering components or centrifuge tube strainers (CTS) of the second vessel are configured to allow flow-through of the ADSCs such that said ADSCs pellet below said CTS, where the second lipo-aspirate is centrifuged through said one or more filtering components, thereby resulting in a majority of the PIF with cell debris above the CTS which ADSCs are below the CTS, and where the PIF within the CTS is removed from the pelleted ADSCs; where the concentrated mesenchymal cell population contain the ADSCs.

In one aspect, the physiological infiltration fluid is saline, Ringer's solution or lactated Ringer's solution. In another aspect, the tubular device is a liposuction cannula having a diameter of between about 2 mm and 6 mm. In one aspect, the diameter is between about 3 mm and 4 mm.

In one aspect, the one or more first filters or CTS have a pore size of about 10 to 250 microns. In a related aspect, the one or more first filters are composed of a material selected from the group consisting of glass fiber, polyester fiber, plastic fiber, metal fiber, composite cellulose and synthetic fiber, nylon mesh, polyester mesh, synthetic fabric, and combinations thereof.

In another aspect, waste material is collected in one or more reservoirs in fluid communication with the vessels.

In one aspect, the first vessel is configured to be deposited on a platform of an orbital shaker, and further including subjecting the first lipoaspirate to rotational force. In a further aspect, the first vessel and second vessel are configured to be deposited in a centrifuge, and further including subjecting said first and second lipoaspirate to hydrodynamic force. In a related aspect, the second one or more filters have a pore size of less than about 50 microns.

In a further related aspect, the second one or more filters are composed of a material including glass fiber, polyester fiber, plastic fiber, metal fiber, composite cellulose and synthetic fiber, nylon mesh, polyester mesh, synthetic fabric, and combinations thereof.

In another aspect, the second vessel is in fluid communication with an output reservoir or a sample chamber.

In embodiments, a method of treating a subject in need thereof with an enriched stromal cells or stem cell population from adipose tissue is disclosed including injecting a physiological infiltration fluid (PIF) into a subject in one or more areas where fat is to be removed; mechanically dissociating fatty tissue in the infiltrated area with a tubular device and removing the dissociated fatty tissue via aspiration; collecting a first lipo-aspirate in a first vessel in fluid communication with the tubular device, where the first vessel includes one or more first filters, and where the filters are configured to retain adipocyte globules and/or aggregates, thereby enriching the mesenchymal cell population of the resulting second lipo-aspirate; concentrating the mesenchymal cell population of the second lipo-aspirate in a second vessel in fluid communication with the first vessel via filter-centrifugation, where one or more filtering components or centrifuge tube strainers (CTS) of the second vessel are configured to allow flow-through of the ADSCs such that said ADSCs pellet below said CTS, where the second lipo-aspirate is centrifuged through said one or more filtering components or CTS, thereby resulting in a majority of the PIF with cell debris above the CTS which ADSCs are below the CTS, and where the PIF within the CTS is removed from the pelleted ADSCs; and administering the mesenchymal cells in the sample reservoir to the subject.

In other embodiments, an enriched fraction of adipocyte derived stem cells (ADSC) isolated by the method above is disclosed, where in the absence of enzymatic dissociating agents endogenous α1β1 integrin increases the expression of collagen expression in the cells. In a related aspect, endogenous α2β1 integrin decreases the expression of MMP-1 (collagenase) when compared to ADSC obtained using enzymatic dissociating agents.

In another aspect, the ADSC cells express one or more cell surface markers includes, but are not limited to, CD9 (tetraspan), CD10 (CALLA), CD13 (aminopeptidase), CD29 (β-1 integrin), CD44 (hyaluronate receptor or phagocytic glycoprotein-1), CD49d (α-4 integrin), CD49e (α-5 integrin), CD51 (α-V integrin), CD54 (ICAM-1), CD55 (DAF), CD59 (complement protectin), CD71 (transferrin receptor), CD73 (5′ nucleotidase), CD90 (Thy-1), CD105 (Endoglin), CD117 (c-Kit), CD146 (Muc18), CD166 (ALCAM), α-smooth muscle actin, Collagen type I, Collagen type II, HLA-ABC, Osteopontin, Osteonectin, or Vimentin.

### BRIEF DESCRIPTION OF THE DRAWINGS

The foregoing summary, embodiments, and other aspects of the present invention will be best understood with reference to a detailed description of embodiments, which follows, when read in conjunction with the accompanying drawings. In the drawings, closely related figures have the same number.

FIG. 1 shows a flow diagram of ADSC enrichment as described.

FIG. 2 shows an illustration of a stacked centrifuge tube strainer (CTS).

FIG. 3 shows an illustration of a CTS within a centrifuge tube or syringe.

FIG. 4 shows an illustration of a CTS within a centrifuge tube or syringe where a sample of fatty tissue has been processed as disclosed herein.

### DETAILED DESCRIPTION OF THE INVENTION

Before the present compositions, methods, and methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “cell” includes one or more cells, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure. All references disclosed herein are incorporated by reference in their entireties.

As used herein, “stem cells” means biological cells found in all multicellular organisms, that can divide through mitosis and differentiate into diverse specialized cell types and can self renew to produce more stem cells. In mammals, there are two broad types of stem cells: embryonic stem cells that are isolated from the inner cell mass of blastocysts, and adult stem cells that are found in various tissues. In adult organisms, stem cells and progenitor cells act as a repair system for the body, replenished in adult tissues. In a developing embryo, stem cells can differentiate into all the specialized cells, but also maintain the normal turnover of regenerative organs, such as blood, skin, or intestinal tissues.

As used herein, “adult stem cells” means undifferentiated cells, found throughout the body after embryonic development, that multiply by cell division to replenish dying cells and regenerate damaged tissues. Also known as somatic stem cells, they can be found in juvenile as well as adult animals and humans.

As used herein, “mesenchymal stem cells (MSCs)” means cells of stromal origin that may differentiate into a variety of tissues. MSCs have been isolated from placenta, adipose tissue, lung, bone marrow and blood, Wharton's jelly from the umbilical cord, and teeth (perivascular niche of dental pulp and periodontal ligament). MSCs are attractive for clinical therapy due to their ability to differentiate, provide trophic support, and modulate innate immune response.

As used herein, “multipotent” means an ability to develop into a limited number of cell types type of an organism. For example, hematopoietic stem cells are multipotent cells that can produce the various cell types found in blood.

As used herein, “pluripotent” means cells that have the ability to give rise to all of the various cell types of the body, but cannot give rise to extra-embryonic tissues such as the amnion, chorion, and other components of the placenta, and cannot produce a living organism. Pluripotency can be demonstrated by providing evidence of stable developmental potential, to form derivatives of all three embryonic germ layers from the progeny of a single cell and to generate a teratoma after injection into an immunosuppressed mouse. Other indications of pluripotency include expression of genes known to be expressed in pluripotent cells, characteristic morphology and patterns of genomic DNA methylation known to be related to pluripotent epigenetic status.

As used herein, “totipotent” means the ability of a cell to develop into all types of cells including extra-embryonic tissues (e.g., placenta) and to give rise to an entire organism (e.g., a mouse or human).

As used herein, the term “subject” means a cell, tissue, or organism, human or non-human, whether in vivo, ex vivo or in vitro, under observation.

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes. Tissues isolated by the present method may be used a biopsy material in the diagnosis of disease.

As used herein, “enzyme dissociating agents,” including grammatical variations thereof include, but are not limited collagenase, hyaluronidase, DNase, elastase, papain, protease type XIV, and trypsin.

As used herein, “physiological solution” or “physiological infiltration fluid (PIF)” means an isotonic salt solution of about 0.9% w/v of NaCl, about 300 mOsm/L. The solution/fluid salinity may be higher (154 mmol/l) than concentration in human serum (Na⁺ 135-145 mmol/l, Cl⁻ 98-109 mmol/l). A solution/fluid osmolarity of 308 mosmol/l is nearly identical with human blood plasma. For example, physiological solutions/fluids include, but are not limited to, saline, Ringer's solution or lactated Ringer's solution. Further, such solutions/fluids may contain an anesthetic, including but not limited to, Lidocaine, but other local anesthetics may be used. In embodiments, the solution or fluid may contain epinephrine.

As used herein, “liposuction,” also known as lipoplasty or suction-assisted lipectomy, is cosmetic surgery performed to remove unwanted deposits of fat from under the skin, and includes, but is not limited to, Tumescent Technique, Dry Technique, Wet Technique, Super Wet Technique, Ultrasonic (UAL), Vaser (ultrasonic) Liposuction, Power Assisted Liposuction (PAL), Laser Liposuction, Tickle (Nutational Infrasonic Liposculpture) Liposuction, and Body Jet Liposuction. Techniques used in liposuction usually depend upon a surgeon, his/her professional skills and preference.

The stromal compartment of mesenchymal tissues is thought to harbor stem cells that display extensive proliferative capacity and multilineage potential. Often called mesenchymal stem cells or stromal stem cells, these cells have been isolated from several mesodermal tissues including bone marrow, muscle, perichondrium and adipose tissue. Stromal stem cells isolated from various mesodermal tissues share key characteristics, including ability to adhere to plastic to form fibroblastic-like colonies (called CFU-F), extensive proliferative capacity, ability to differentiate into several mesodermal lineages including bone, muscle, cartilage and fat, and express several common cell surface antigens. Recent evidence suggests that these cells can also form non-mesodermal tissues including neuron-like cells.

All of these attributes make mesenchymal stem cells an attractive cell source for use in several clinical applications, including cell based therapies for treatment of disease such a Parkinson's and Alzheimer's diseases, spinal cord injuries, burns, heart disease, and osteoarthritis, among other conditions.

Adipose derived stem cells (ADSCs) may be obtained from fatty tissue harvested through liposuction (termed processed lipoaspirate cells (PLAs)), or through abdominoplasty procedures. The identification of ASC cells in physiological infiltration fluid (PIF) is well known (see, e.g., Gimble et al. Circulation Res (2007) 100:1249-1260 and Yoshimura et al. J Cell Physiol (2006) 208:64-76). The ADSC cells are separated from the fat globules at the time of liposuction as a result of physical dissociation. Steps are disclosed, as described herein, to enhance the process of physical dissociation of cell and then simplify and expedite ADSC isolation from the fat globules.

### Wet Liposuction

During wet liposuction method a certain amount of fluid PIF is injected before the procedure. This fluid consists of intravenous salt solution, a local anesthetic substance lidocaine and epinephrine (i.e., adrenaline), a medication that make the blood vessels contract. This fluid is injected into the areas where fat is to be removed.

Injection of medical solutions has many advantages and is used by many plastic surgeons. Due to these techniques more fat can be liposuctioned more easily. During this method of liposuction less blood is lost and anesthesia is provided for the plastic surgery. Due to fluid injection, bruising is less visible after surgery.

During wet technique an incision is made on the skin. Then a surgeon inserts a tiny metal tube under the skin into a fatty tissue. This tube is moved around while infusing PIF into the fatty tissue. Afterwards, fatty deposits are suctioned with the help of a vacuum pump or syringe attached to a liposuction cannula.

### Super Wet Liposuction

The difference between a wet and super-wet technique is in the amount of physiological solution injected. During super-wet technique the amount of fluid injected prior to liposuction is usually equal to the amount of fat to be removed. During wet technique a smaller amount of fluid is injected.

In the tumescent technique for liposuction, a large volume of very dilute solution of local anesthesia (lidocaine and epinephrine) is infiltrated (injected) into the fat beneath the skin, causing the targeted area to become tumescent. Large amounts of fluid used inflate or “tumesce” the fat compartments making them swollen and firm. The expanded compartments permit the liposuction cannula to pass under the skin smoothly as fat is removed.

The local anesthetic lidocaine in the tumescent solution provides such complete local anesthesia, that it eliminates the need for general anesthesia, or IV sedation. The drug epinephrine (adrenaline) provides localized vasoconstriction that virtually eliminates surgical bleeding during tumescent liposuction. By eliminating the risks of general anesthesia and the risks of excessive surgical bleeding, the tumescent technique for liposuction has effectively eliminated the many of the dangers associated with the older forms of liposuction (e.g., as blood loss is minimized there is a lower chance to need a blood transfusion after surgery).

Injected amounts vary depending on what needs to be done. Sometimes the injected amount is as much as three times the amount of fat to be removed. Tumescent liposuction is typically performed on patients who need only a local anesthetic. This surgery can take significantly longer than traditional liposuction (and sometimes as long as several hours).

After the patient receives intravenous sedation medication, the incision sites are injected with a thin needle. A scalpel then makes a tiny hole. A larger infiltration cannula then is used to introduce the anesthetic solution into the entire region to be sculpted.

Fluid is injected using the various incisions for the liposuction. The infiltration continues until the tissues are expanded with the fluid (or tumescent).

Adipose tissue is an abundant source of mesenchymal stem cells, which have shown promise in the field of regenerative medicine. Furthermore, these cells can be readily harvested in large numbers with low donor-site morbidity. During the past decade, numerous studies have provided preclinical data on the safety and efficacy of adipose-derived stem cells, supporting the use of these cells in future clinical applications. Various clinical trials have shown the regenerative capability of adipose-derived stem cells in subspecialties of medical fields such as plastic surgery, orthopedic surgery, oral and maxillofacial surgery, and cardiac surgery. In addition, a great deal of knowledge concerning the harvesting, characterization, and culture of adipose-derived stem cells has been reported.

Typically, initial enzymatic digestion of adipose tissue yields a mixture of stromal and vascular cells referred to as the stromal-vascular fraction (SVF). A putative stem cell population within this SVF named processed lipoaspirate (PLA) cells.

There is no consensus when it comes to the nomenclature used to describe progenitor cells from adipose tissue-derived stroma, which can sometimes lead to confusion. The term PLA refers to adipose-derived stromal cells and adipose-derived stem cells (ASCs) and has often been used to describe cells obtained immediately after collagenase digestion. ASCs exhibit stable growth and proliferation kinetics and can differentiate toward osteogenic, chondrogenic, adipogenic, myogenic, or neurogenic lineages in vitro. Furthermore, a group has recently described the isolation and culture of ASCs with multipotent differentiation capacity at the single-cell level.

### Characterization and Localization

Adipocyte-Derived Stem Cells (ADSCs) express the mesenchymal stem cell markers CD10, CD13, CD29, CD34, CD44, CD54, CD71, CD90, CD105, CD106, CD117, and STRO-1. They are negative for the hematopoietic lineage markers CD45, CD14, CD16, CD56, CD61, CD62E, CD104, and CD106 and for the endothelial cell (EC) markers CD31, CD144, and von Willebrand factor. Morphologically, they are fibroblast-like and preserve their shape after expansion in vitro.

The similarities between ADSCs and BSCs may indicate that ADSCs are derived from circulating BSCs, which infiltrate into the adipose compartment through vessel walls, however, they do not express endothelial or hematopoietic cell markers.

### Paracrine Secretion

Adipose tissue actively participates in endocrine processes by secreting cytokines and growth factors. ADSCs secrete high levels of epidermal growth factor (EGF), vascular endothelial growth factor (VEGF), basic fibroblast growth factor (bFGF), keratinocyte growth factor (KGF), platelet-derived growth factor (PDGF), hepatocyte growth factor (HGF), transforming growth factor-beta (TGF-β), insulin-like growth factor (IGF), and brain-derived neurotrophic factor (BDNF). They also secrete cytokines such as Flt-3 ligand, granulocyte colony stimulating factor (G-CSF), granulocyte/macrophage colony stimulating factor (GM-CSF), macrophage colony stimulating factor (M-CSF), interleukin-6 (IL-6), interleukin-7 (IL-7), interleukin-8 (IL-8), interleukin-11 (IL-11), interleukin-12 (IL-12), leukemia inhibitory factor (LIF), and tumor necrosis factor-alpha (TNF-α). This secretion of paracrine factors by the adipose tissue likely contributes to the elevated levels of these cytokines in cases of obesity.

It is important to note that these angiogenic and anti-apoptotic growth factors are secreted in bioactive levels by ADSCs and that their secretion increases significantly under hypoxic conditions. HGF is possibly the main angiogenic factor secreted by ADSCs and it plays a central role in the paracrine effects on ADSCs. Its suppression has been shown to impair the angiogenic and regenerative effects of ADSCs in ischemic tissues. Silencing HGF reduces the ability of ASCs to promote EC proliferation and inhibits the pro-angiogenic effects of HGF in vitro.

### Soft Tissue Regeneration and Wound Healing

The materials currently used in soft tissue regeneration, which include collagen, hyaluronic acid, silicon, and other filler materials, have several disadvantages such as high cost, immunogenicity/allergenicity, and the risk of transmitting infectious diseases. Meanwhile, autologous fat grafts are more widely available; however, one limitation of this technique is the poor long-term graft retention in current clinical practice. The transplanted fat grafts can lose volume over time due to tissue resorption that can result in the loss of 20-90% of the original graft volume. The ideal solution for soft tissue regeneration would promote the regeneration of vascularized adipose tissue to completely fill the defect volume.

Compared with transplanted fat tissue alone, fat tissue transplanted with non-cultured ADSCs had a higher density of capillaries six and nine months after transplantation (see, e.g., Tobita et al., Discov Med (2011) 11(57):160-170). The reasons for these successful results might be the pro-angiogenic growth factors secreted by ADSCs, as described previously.

Wound healing might be interrupted by a variety of pathological conditions, such as diabetes, radiation and immunosuppression, resulting in refractory chronic wounds. Growth factors involved in wound healing have been individually applied to the wound to promote wound healing in unfavorable conditions. However, the theoretical promise of this approach was unfulfilled due to the complex nature of wound healing, which involves a number of different growth factors. To achieve optimal results, all these growth factors should be applied continuously, as opposed to the intermittent applications of individual growth factors.

ADSCs secrete nearly all of the growth factors that take part in normal wound healing. After application, ADSCs may remain viable at the wound site and secrete growth factors in a continuous and regulated manner in response to environmental cues, just as occurs in the natural wound healing process. ADSCs may promote wound healing by increasing vessel density, granulation tissue thickness, and collagen deposition, and they may also improve the cosmetic appearance of resultant scars.

A ready blood supply is crucial for wound healing. VEGF secreted from ADSCs induces the migration and proliferation of ECs, increasing the vascularity of the wound bed.

### Musculoskeletal Regeneration

Current therapeutic approaches for muscle loss cannot restore muscle function effectively. ADSCs can differentiate into chondrogenic, osteogenic, and myogenic cells in vitro, and thus could potentially be used to regenerate tissue in musculoskeletal system disorders.

Muscle tissue contains muscle progenitor cells called satellite cells that lie underneath the basal lamina. These cells can divide and fuse to repair or replace damaged fibers in response to acute muscle injury or in chronic degenerative myopathies. However, continuous muscle degeneration-regeneration cycles in chronic cases lead to a depletion of the satellite cell pool. Moreover, it is difficult to expand satellite cells in vitro and they rapidly undergo senescence.

ADSCs may provide an easily accessible and expandable alternative cell source for the cellular therapy of muscular disorders.

ADSCs can form osteoid in vitro and in vivo. ADSCs may be combined with biomaterials to successfully repair critical bone defects. Moreover, ADSCs secrete osteoinductive growth factors, which may potentially recruit host bone-forming cells and induce osteogenesis when implanted in vivo. ADSCs genetically modified to secrete bone morphogenic protein-2 (BMP-2) may also be an effective method for enhancing bone healing.

### Cardiovascular Regeneration

Acute and chronic ischemic heart diseases are among the leading causes of mortality worldwide. Conventional management generally does not replace lost cardiomyocyte mass or myocardial fibrotic tissue. The injection of both cultured and freshly-isolated ADSCs has the potential to improve cardiac function in experimentally-induced myocardial injury.

### Nervous System Regeneration

A variety of growth factors, such as nerve growth factor (NGF), ciliary neurotrophic factor (CNF), IGF, and FGF, are secreted from nerve stumps following injury. These growth factors stimulate axonal growth in close contact with Schwann cells, which are the primary support cells of the peripheral nervous system. Since the above-mentioned regeneration sequence is known to fail in long nerve defects, cellular treatments, such as stem cell therapy, might be useful as a means to introduce growth factors into the gap and thereby promote nerve regeneration. ADSCs can secrete some nerve growth factors, including IGF and FGF, so these cells might have the capacity to promote nerve healing.

It has been well established that ADSCs can survive in the nervous system after injection and promote nerve healing either by direct differentiation or through the secretion of a number of paracrine factors. ADSCs thus show promise for the future of the treatment of central nervous system injuries, as well as peripheral nerve injuries.

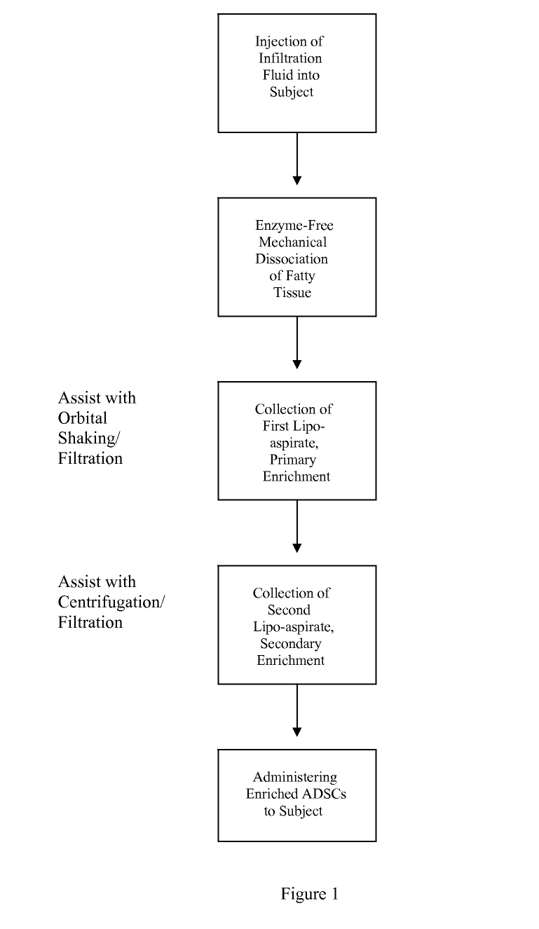

In embodiments, a method of enriching a stromal cell population or stem cell population from adipose tissue in the absence of a dissociating reagent is disclosed, including injecting a PIF into a subject in one or more areas where fat is to be removed; mechanically dissociating fatty tissue in the infiltrated area with a tubular device and removing the dissociated fatty tissue via aspiration; collecting a first lipo-aspirate in a first vessel in fluid communication with the tubular device, where the first vessel comprises one or more first filters, and where the filters are configured to remove adipocyte globules/aggregates, thereby enriching the mesenchymal cell population of the resulting second lipo-aspirate; and concentrating the mesenchymal cell population of the second lipo-aspirate in a second vessel in fluid communication with the first vessel via filter-centrifugation, where one or more filtering components or centrifuge tube strainers (CTS) of the second vessel are configured to allow flow-through of the ADSCs such that said ADSCs pellet below said CTS, where the second lipo-aspirate is centrifuged through said one or more filtering components or CTS, thereby resulting in a majority of the PIF with cell debris above the CTS which the ADSCs are below the CTS, and where the PIF within the CTS is removed from the pelleted ADSCs; where the concentrated mesenchymal cell population comprises the ADSCs (see FIG. 1).

In embodiments, stromal cells may be isolated from lipoaspirate collected from several regions of the body including, but not limited to, hip, thigh and abdominal regions. The amount of fat liposuctioned depends on the number of ADSCs needed or the volume of fat needed for lipo-augmentation. The latter can vary from 5 to 15 ml for the face to 600 to 1000 ml needed for buttock lipo-augmentation. Thus, the volume of PIF used may vary, and may be selected as determined by one of skill in the art. For buttock lipo-augmentation, for example, commonly about 5000 ml of PIF may be used for infiltration.

In one aspect, at least about 300 to about 2500 ml of lipo-aspirate may be collected, wherein about 1.8×10¹² stromal cells/ml may be obtained from the PIF by the methods as described. In a related aspect, the total number of stromal cells obtained from fat globules treated with collagenase is about 2.4×10⁹ cells/ml.

In one aspect, such isolated ADSCs may express one or more of the following stem cell surface markers: CD9 (tetraspan), CD10 (CALLA), CD13 (aminopeptidase), CD29 (β-1 integrin), CD44 (hyaluronate receptor or phagocytic glycoprotein-1), CD49d (α-4 integrin), CD49e (α-5 integrin), CD51 (α-V integrin), CD54 (ICAM-1), CD55 (DAF), CD59 (complement protectin), CD71 (transferrin receptor), CD73 (5′ nucleotidase), CD90 (Thy-1), CD105 (Endoglin), CD117 (c-Kit), CD146 (Muc18), CD166 (ALCAM), α-smooth muscle actin, Collagen type I, Collagen type II, HLA-ABC, Osteopontin, Osteonectin, or Vimentin. The undifferentiated ADAS cells of the present disclosure may be homogeneously positive for the cell-surface markers CD10, CD13x, CD29, CD44, CD49e, CD59, CD90, and HLA-ABC, and homogeneously negative for the cell surface markers CD11b, CD45, and HLA-DR. The absence of the panhematopoietic marker, CD45, indicates that the ADAS cells do not derive from circulating BM hematopoietic stem cells.

As stated above, the methods of the present disclosure are conducted in the absence of proteases or other enzymatic dissociation agents. Among other things, enzymatic treatment prolongs the process of ADSC isolation and purification while the enzyme breaks down ECM components, such as collagen. In addition, such enzymatic treatment may have adverse effects on adipocyte viability, which is an important aspect to keep in mind when adipocyte and ADSC are used together during the process of lipoaugmentation. The process, as described herein, offers efficient and rapid isolation of cells that may be used immediately at the time of surgery, (e.g., the time required for enzymatic digestion is eliminated).

As demonstrated by Mondeh et al. (Millipore Newsletter, Collagenase Type I for Enzymatic Passaging of Human Embryonic Stem Cells in HEScGro™ Medium), depending on the type of dissociating agent used, some commonly applied enzymes (e.g., collagenase type IV) may cause stem cells to differentiate. Similarly, evidence has been provided by Pilgaard et al. (Regen Med (2008) 3(5):705-715), showing that consistent isolation of ADSCs is particularly dependent on the protease formulation. By circumventing the use of such agents, the ADSCs of the present disclosure do not suffer from inconsistencies associated with enzyme treated adipose tissue aspirates with respect to nucleated cells, viability and frequency of specific lineages (see, e.g., Pilgaard et al. (2008)).

While not being bound by theory, it is well known in the art that ECM plays a vital role in determining the fate of stem cells (see, e.g., Guilak et al., Cell (2009). As such, it is logical to conclude that the addition of enzymes as dissociation agents, at minimum, qualitatively change the ECM environment as many of the constituents of the ECM will be degraded by the enzymes typically used in dissociation of fatty tissues (e.g., collagenases; see Pilgaard et al. (2008)). As such, the ADSCs of the present disclosure would at least contain on their surfaces a different “landscape” of intact substrates that would normally be degraded by said enzymatic dissociation agents, and thus, would allow for detection of assayable distinctions between the ADSC cells of the present disclosure and those isolated by means which employ such enzymatic agents.

For example, the main target of collagenase, of course, is collagen, which is the most abundant protein in the ECM. Salasznyk et al. (J Biomed Biotech (2004) 1:24-34) has demonstrated that osteogenic differentiation occurs in hMSC when plated on vitronectin and collagen I, where almost no differentiation took place with fibronectin or on non-coated plates. In fact, Lagholz et al. (J Cell Biology (1995) 131 (6, Pt. 2):1903-1915) have shown that changes in integrin-collagen interaction change gene expression patterns in cells. Together, these data support the conclusion that enzymatic disassociation of cells (1) affects the ECM and (2) is capable of changing the surface of cells so treated, where in fact such changes can be measured biochemically (e.g., changes in gene expression) or functionally (e.g., cell viability, lineage stability, and differentiation). In one aspect, ADSCs isolated by the disclosed method are described, which cells may be distinguished biochemically and functionally from ADSC obtained using enzymatic dissociating agents.

In embodiments, cells isolated by the disclosed methods may be differentiated from enzymatically treated cells by determining modulatory effects of α1β1 integrin on collagen expression and/or α2β1 integrin induction of MMP-1 (collagenase) expression. Such determining may be carried out using PCR, protein electrophoresis and the like. Alternatively, cells isolated by the present method may be distinguished via function by their competency/efficiency of differentiation into other cell types.

In embodiments, a liposuction device is disclosed for removing subcutaneous fat below a skin surface. Substantially, the liposuction device is formed by a tubular device comprising a cannula, a first vessel, a second vessel, a physiological solution reservoir, optionally a waste vessel, optionally a sample vessel, one or more pumps with allocated fluid conduit lines, a pressure selector and a pump control switch. In embodiments, the tubular device is passed through a small cut opening in the skin and dips, with its front end, into the subcutaneous fat below the surface of the skin.

At one end, the suction cannula contained therein may comprise one or more suction openings at its circumference, through which the mixture of subcutaneous fat and physiological solution is continuously sucked from the region below the skin surface at a negative pressure. In some embodiments, the negative pressure is about 0.5 to about 0.6, about 0.6 to about 0.7, about 0.7 to about 0.8, or about 0.8 to about 0.9 bar. In one aspect, the negative pressure is about 0.5 to about 0.9. In a rear portion, the suction device may comprise a handle which is enlarged with respect to the front portion of the suction lance. In its front portion, the cannula may have an outer diameter of about 2.0 to about 6.0 mm. The free front end of the suction cannula may contain stainless steel or an alloy or combination thereof. The choice of material comprising said cannula may be made by one of skill in the art.

A working fluid pump pumps the PIF from the physiological solution reservoir via a flexible conduit into the injection line of the tubular device. The pump may operate continuously, or may also be operated intermittently, so that the PIF is injected intermittently or pulsatingly. The working fluid may be injected through an injection opening at an overpressure of about 10-100 bar. Thereby, fatty tissue and fat are mechanically detached from the subcutaneous fat, but no blood vessels are destroyed. The PIF may contain pain-killing substances, but may also consist of mere salt solution.

Through a flexible conduit, the suction cannula may be connected with a suction pump generating a negative pressure by which pump the sucked liquid is continuously pumped into one or more vessels.

In embodiments, a pump switch is configured to connect with a control device through a control line. By actuating a pump switch, the PIF pump and the suction pump are switched on, upon release of the pump switch, the PIF pump and the suction pumps may be switched off again. Thus, a simple control of the operation of the liposuction device is possible and it is possible at any time to switch it off quickly in order to avoid undesired removals.

In embodiments, the dissociated lipoaspirate is transferred to a first vessel, where the first vessel comprises one or more filters or membranes, where filtration through the filters or membranes is initiated with any conventional process such as centrifugal force, gravity or constant negative suction pressure to separate the fat globules within the filter from the filtrate—the second lipo-aspirate fluid containing macrophages, stromal cells and endothelial cells (ADSCs-PIF mix). In one aspect, the first vessel is configured to be deposited in a centrifugation rotor or to be deposited on a platform of an orbital shaker. In a related aspect, the centrifugation may be carried out at about 500 to about 1000 rpm, about 500 to about 1200 rpm, about 700 to about 1400 rpm, or about 800 to 1500 rpm. In certain aspects, centrifugation may be carried out at about 600 to 1500 rpm for about 5-10 minutes. In other aspects, centrifugation may be carried out at about 1000 to 1500 rpm for about 5-10 minutes.

In embodiments, this process as disclosed may be applied to small volumes (10 to 50 ml). In other embodiments, the process as disclosed may be applied to larger volumes (e.g., 250 cc to 5000 ml). In one aspect, when larger volumes are used, the process of ASCs-PIF separation is simplified and optimized by use of centrifugation-infiltration. In a related aspect, a filter of about 30 to 50 micron may be used within the centrifuge tube. In one aspect, a filter of about 40 microns may be used.

In embodiments, the centrifuge tubes can range in size from 200 to 1000 ml. For example, in a 50 ml centrifuge tube, the capacity of the varies from about 45 to about 48 ml. In a related aspect, during centrifugation at 600 to 1500 rpm for about 5-10 minutes, ADSCs passes through the filter. In one aspect, by reducing the g-force to about 100 to 600 rpm for about 5 to 10 minutes, while the ADSCs are centrifuged through the filter, the majority of the red blood cells and debris remain within the strainer/filter.

In embodiments, the rotational speed of the orbital shaker is about at about 50 to about 250 rpm, about 60 to about 200 rpm, about 80 to about 150 rpm, about 90 to 100 rpm. In a related aspect, for 1 liter of lipoaspirate, about 10 to about 20 minutes of rotational agitation may be used in order to separate the ADSC-PIF from the fat globules.

Typically, nominal pore sizes of theses one or more filters or membranes of the first vessel range from about 10 microns to about 250 microns, about 20 microns to about 200 microns, about 30 microns to about 100 microns, about 40 to about 50 microns.

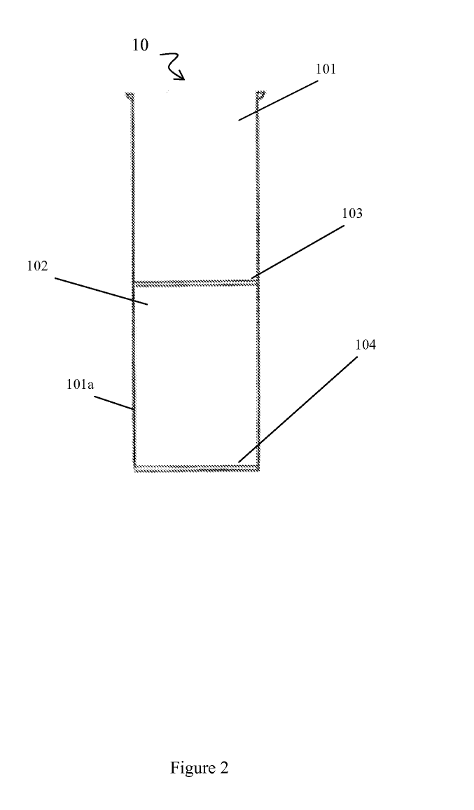

The filters or membranes or centrifuge tube strainers (CTS) of the first vessel may be formed from any natural or synthetic polymers, paper, ceramics or metals such as stainless steel or nickel. In one aspect, the CTS comprises one or more plastic frames and filter material (see, e.g., FIGS. 2-4).

Other embodiments for said filters or membranes or CTS include, but are not limited to, nitrocellulose, regenerated cellulose cellulose acetate, polysulphones including polusulphone, polyestersulphone, polyphenolsulphones and polyarylsulphones, polyvinylidene fluoride, polyolefins such as ultrahigh molecular weight polyethylene, low density polyethylene and polypropylene, nylon and other polyamides, PTEF, thermoplastic fluorinated polymers, polycarbonates. All of these membranes are well known in the art and are commercially available from a variety of sources including Millipore Corporation of Bedford, Mass.

In embodiments, the filters or membranes or CTS may be treated to contain antibodies/ligands which recognize select surface moieties that are specific to select cell types. For example, endothelial cells (CD31⁺) and leukocytes (CD45⁺) may be bound to said filters using anti-CD31⁺ and/or anti-CD45⁺ antibodies. Unwanted cells (CD31⁺/CD45⁺) are retained on the filters and/or membranes and unbound CD31⁻ and CD45⁻ stem cells pass through the filters and/or membranes.

In embodiments, the first lipoaspirate is transferred to a second vessel, where the second vessel comprises one or more filters or membranes or CTS, and where the second vessel is configured to be deposited in a centrifugation rotor.

Typically, nominal pore sizes of theses one or more filters or membranes or CTS range from less than about 10 microns or about 10 microns to about 50 microns. The filters or membranes or CTS of the second vessel may also be formed from any natural or synthetic polymers, paper, ceramics or metals such as stainless steel or nickel. Other embodiments include, but are not limited to, nitrocellulose, regenerated cellulose cellulose acetate, polysulphones including polusulphone, polyestersulphone, polyphenolsulphones and polyarylsulphones, polyvinylidene fluoride, polyolefins such as ultrahigh molecular weight polyethylene, low density polyethylene and polypropylene, nylon and other polyamides, PTEF, thermoplastic fluorinated polymers, polycarbonates. All of these membranes are well known in the art and are commercially available from a variety of sources including Millipore Corporation of Bedford, Mass. In certain aspects, in addition to retaining unwanted cells (including erythrocytes), the filters or membranes remove collagen, free lipids, and excess fluid.

In embodiments, the filters or membranes or CTS of the second vessel may also be treated to contain antibodies/ligands which recognize select surface moieties that are specific to select cell types. For example, endothelial cells (CD31⁺) and leukocytes (CD45⁺) may be bound to said filters using anti-CD31⁺ and/or anti-CD45⁺ antibodies. Unwanted cells (CD31⁺/CD45⁺) are retained on the filters and/or membranes and unbound CD31⁻ and CD45⁻ stem cells pass through the filters and/or membranes.

In embodiments, once the enriched mesenchymal population of cells is obtained, such cells may be administered into subjects in need thereof. In one aspect, before administration cells may be washed and transferred into a specialized centrifuge tube for pelleting. The cell pellets are then gently reconstituted in a small volume of lactated ringer. The cells are then aspirated into a syringe for use. In a related aspect, the cell pellet is reconstituted in about 1 to about 10 ml of lactated ringer for use. In another aspect, ADSCs may be recombinantly transformed before administration. In embodiments, all materials (e.g., filters, tubes, cannulas, vessels and the like) may be autoclavable and/or disposable.

Those of skill in the art will recognize that many of the functions and aspects of such a method may be implemented on a computer or computers. The hardware of such computer platforms typically is general purpose in nature, albeit with an appropriate network connection for communication via the intranet, the Internet and/or other data networks.

As known in the data processing and communications arts, each such general-purpose computer typically comprises a central processor, an internal communication bus, various types of memory (RAM, ROM, EEPROM, cache memory, etc.), disk drives or other code and data storage systems, and one or more network interface cards or ports for communication purposes. The computer system also may be coupled to a display and one or more user input devices such as alphanumeric and other keys of a keyboard, a mouse, a trackball, and the like. The display and user input element(s) together form a service-related user interface, for interactive control of the operation of the computer system. These user interface elements may be locally coupled to the computer system, for example in a workstation configuration, or the user interface elements may be remote from the computer and communicate therewith via a network. The elements of such a general-purpose computer system also may be combined with or built into routing elements or nodes of the network.

The software functionalities (e.g., many of the operations described above) involve programming of controllers, including executable code as well as associated stored data. The software code is executable by the general-purpose computer that functions as the particular computer. In operation, the executable program code and possibly the associated data are stored within the general-purpose computer platform. At other times, however, the software may be stored at other locations and/or transported for loading into the appropriate general-purpose computer system. Hence, the embodiments involve one or more software products in the form of one or more modules of code carried by at least one machine-readable medium. Execution of such code by a processor of the computer platform enables the platform to implement the system or platform functions, in essentially the manner performed in the embodiments discussed and illustrated herein.

As used herein, terms such as controller or CPU or computer or machine readable medium refer to any medium that participates in providing instructions to a processor for execution. Such a medium may take many forms, including but not limited to, non-volatile media, volatile media, and transmission media. Non-volatile media include, for example, optical or magnetic disks, such as any of the storage devices in any computer(s). Volatile media include dynamic memory, such as main memory of such a computer platform. Physical transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD, any other optical medium, punch cards, paper tape, any other physical medium with patterns of holes, a RAM, a PROM, and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution. In embodiments, the device of the present disclosure may be combined with a CPU containing a microprocessor in electro-mechanical communication with the valves, pumps and gauges of said device

In embodiments, the method as disclosed may be carried out using a kit, which kit may include a liposuction device containing a tubular device comprising a cannula, a first vessel, a second vessel, a PIF reservoir, optionally a waste vessel, one or more filters or membranes or CTS, optionally a sample vessel, one or more pumps with allocated fluid conduit lines, a pressure selector and a pump control switch, labels, buffers, and instructions/manuals on how to use such materials, where such instructions/manuals may be provided on tangible materials such as pamphlets, CDs, or DVDs and/or through links to internet sites.

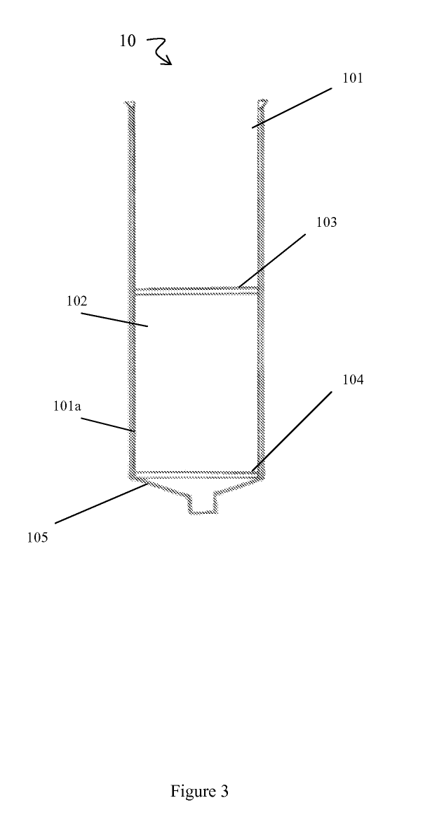

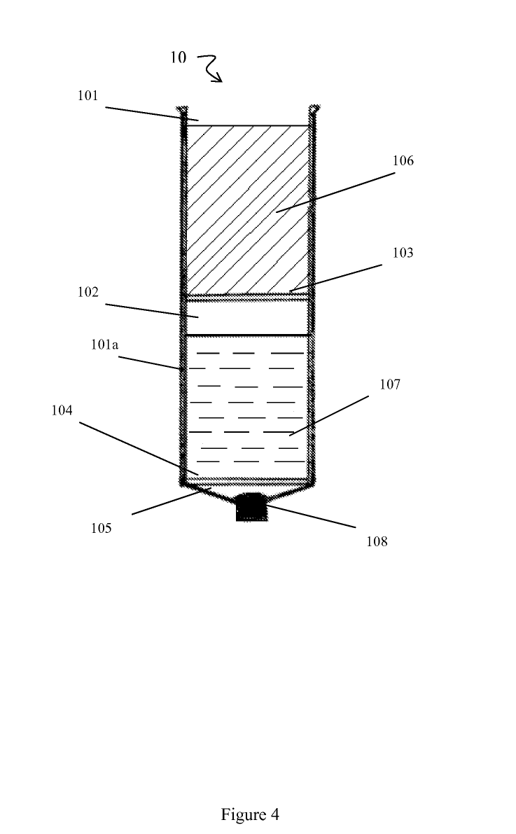

In embodiments, a specialized stacked CTS 10 harbors two filters 103,104 (FIG. 2). The top filter 103 will be in the range of about 10 to about 250 microns. In one aspect, the top filter 103 may be about 125 microns. In a related aspect, the lower filter 104 may be less than about 50 microns. In one aspect, the lower filter 104 may be about 25 microns. The two filters 103,104 are within a tubular structure 101a which may be placed within a corresponding centrifuge tube/syringe 105 of similar size (FIGS. 3 and 4). The chamber 101 over the top filter 103 “termed first chamber” resembles the first vessel. The chamber 102 in between the two filters 103,104 “termed the second chamber”, resembles the second vessel. During processing, a first lipoaspirate may be placed within the stacked CTS/centrifuge tube 10/105 (FIG. 4). Centrifugation of the tube 105 at about 500 to about 1500 rpm retains the fat globules 106 over the top filter 103 while the ADSCs-PIF mix 107 passes through onto the lower filter 104. An ADSC pellet 108 may be formed under the lower filter 104. Fat globules 106 may be harvested over the top filter 103 while the PIF 107 is removed, trapped in between the 2 filters 103,104.

In embodiments (e.g., FIG. 4), the stacked CTS 10 may have a capacity of about 48 ml and may be placed snuggly within a 50 cc centrifuge tube 105. The first chamber 101 may have a capacity of about 25 ml. Twenty five ml of the first lipoaspirate may be placed over the top filter 103. The centrifuge tube 105 may then spin at about 1200 rpm for about 5 to about 10 minutes. During centrifugation, ADSC-PIF mix 107 passes through the top filter 103 into the second chamber 102. The second chamber 102 may have a capacity of about 23 ml. Continued centrifugation results in migration of the ADSC cells through the lower filter 104. Fat globules 106 will be over the first filter 103 while the ADSC pellet 108 will be under the second filter 104. In one embodiment, capped syringes may be used in place of said centrifuge tube 105.

The following examples are intended to illustrate but not limit the invention.

### EXAMPLES

### Identification of ADSC Cells in Physiological Infiltration Fluid

Briefly, prior to liposuction, the subject's subcutaneous fat is infiltrated with a physiological infiltration fluid (PIF). To optimize fat cell dissociation, for every 1 cc of fat planned for liposuction, 3-5 cc of the physiological solution is used for infiltration. This ratio of PIF enhances the process of physical dissociation of fat from ADSCs. The amount of fat liposuctioned depends on the number of ADSCs needed or the volume of fat needed for lipo-augmentation. The latter can vary from 5 to 15 cc of fat for the face to 600 to 1000 cc needed for buttock lipo-augmentation. Thus, the volume of physiological infiltration fluid (PIF) used will depend on the area selected. Next, during liposuction, about 300 to 2500 cc of lipoaspirate was suctioned using a 3 mm liposuction cannula.

Separation of ADSCs from Fat Globules

During liposuction, the lipo-aspirate (containing fat globules, ADSC cells, PIF, blood cells and cell debris) was collected in a sterile liposuction canister of 1 to 3 liters that harbors a nylon filter of 10 to 250 microns. At this range, the majority of the fat globules were retained within the filter while the ASC-PIF mix passed through the pores.

To optimize ADSC-PIF separation from the fat globules, and to expedite the process, an orbital shaker that rotates at about 50 to about 250 rpm was employed. The orbital shaker agitates the nylon filter within the canister. The agitation expedites migration of the ADSC-PIF through the filter. In this manner, a more pure concentration of fat globules may be obtained (for later fat transfer). For 1 liter of lipoaspirate, approximately 20 minutes of rotational agitation was used in order to separate the ADSC-PIF from the fat globules. The orbital shaker functions throughout the process of liposuction, expediting the process of separation.

### Cell Count

Cell count in 1 cc of fat globules as well as 1 cc of PIF was determined using a hemocytometer. Fat globules were treated with collagenase according to conventional methods (see, e.g., Sardjano et al., Med J Indones (2009)18(2):91-96). The sample was then made up to 10 cc using phosphate buffered saline (PBS). ADSC cells were then centrifuged at 300 g for 5 minutes in a centrifuge tube. The cell pellet was then reconstituted in 1 cc of PBS. Similarly, the PIF sample was made up to 10 cc using PBS. The sample was then centrifuged, and the ADSC pellet reconstituted in 1 cc of PBS. Cell count was then performed in the hemocytometer according to the manufacturer's instructions. Final total sample counts were then established by considering the dilution factor for each sample, i.e. the total volume of fat globules and PIF.

| | Total number of stromal | Total number of stromal |
|---|---|---|
| Sample | cells/ml | cells per sample |
| Fat globules | 2.41 × 10⁹ | 1.807 × 10¹² |
| PIF-ADSCs | 3.88 × 10¹⁰ | 1.164 × 10¹⁴ |

Separation of Adipocyte Stromal Cells from Physiological Fluid-Open System

The ADSCs were then separated from the physiological infiltration fluid by centrifugation at 1000 to 1500 rpm for 5-10 minutes. Although, this process works well for smaller volumes (10 to 50 cc), for larger volumes (e.g., 250 cc to 5000 cc) a large percentage of the cells are lost during aspiration/decanting of the infiltration fluid. To overcome this problem, the process of ADSCs-PIF separation was simplified and optimized by use of centrifugation-infiltration. During this process, a filter of about 10 to 50 micron was used within the centrifuge tube. The centrifuge tubes can range in size from 200 to 1000 cc.

During centrifugation at 600 to 1500 rpm for 5-10 minutes, ADSCs passed through the filter. The PIF was then removed from the stromal cells within the filter. In this fashion, the infiltration fluid containing fluid, red blood cells, oil, and broken cell debris was separated from the centrifuged ADSCs. By reducing the g-force to about 100 to 600 rpm for 5 to 10 minutes, while the ADSCs are centrifuged through the filter, the majority of the red blood cells and debris remain within the strainer. This is of importance, since the ADSC viability is inversely related to the number of the red blood cells within the final fraction of ADSCs that is to be used for tissue augmentation.

### Washing of ADSCs

Centrifugation-infiltration may also be used to optimize washing of the ADSCs with a wash fluid. This solution may be composed of lactated ringer, or phosphate buffer saline, or a cell culture medium such as DMEM. Following the centrifugation-infiltration, the PIF is decanted/aspirated off of the filter. The wash fluid is then poured over the filter. After a period of 5 minutes, centrifugation-infiltration is repeated again. The wash fluid is then removed within the filter.

An alternative approach includes reconstitution of the ADSC cells with wash fluid after the first centrifugation-infiltration. The cells are then placed in a second tube and centrifuged through the filter again.

Separation of Adipocyte Stromal Cells from Physiological Fluid-Closed System

The components of the open system may be assembled in such as way as to devise a partial or complete closed system. In the closed system, a transfer tube (TT) is connected the PIF collection canister directly to a specialized centrifuge tube (SCT). Once PIF is transferred to the centrifuge tube, centrifugation is initiated for 5-10 minutes at 300 g. At the completion of centrifugation, the PIF with cell debris are gently aspirated from the centrifuge tube through the same TT. At this stage, the cells may be washed with a wash media that is connected to the TT. Following transfer of wash fluid into the specialized centrifuge tube, centrifugation is repeated. The wash fluid is then aspirated through the TT. The cell pellets are then gently reconstituted in a small volume of lactated ringer. The cells are then aspirated into a syringe for use.

In a partial closed system, the PIF is transferred into the specialized centrifuge tube via TT. The SCT can harbor one or more centrifuge tube strainers (CTS). The SCT is placed within its centrifuge bucket. Following completion of centrifugation at 50-300 g for 5-10 minutes, the ADSC cells are concentrated below the CTS. The PIF is then aspirated through the TT. Washing is then performed if needed. Following completion of the wash process, the SCT is then removed from its bucket and disassembled under sterile conditions. The cell pellet(s) are then reconstituted in 1-10 cc of lactated ringer for use.

Although the invention has been described with reference to the above examples, it will be understood that modifications and variations are encompassed within the spirit and scope of the invention. Accordingly, the invention is limited only by the following claims. All references cited herein are incorporated by reference in their entireties.

We claim herein:

1. A method of enriching stromal cell and stem cell populations from adipose tissue in the absence of a dissociating reagent comprising:
a) injecting an physiological infiltration fluid (PIF) into a subject in one or more areas where fat is to be removed;
b) mechanically dissociating fatty tissue in the infiltrated area with a tubular device and removing the dissociated fatty tissue via aspiration;
c) collecting a first lipo-aspirate in a first vessel in fluid communication with said tubular device, wherein said first vessel comprises one or more first filters, and wherein said filters are configured to retain adipocyte globules and/or aggregates, thereby removing fat from infiltration fluid comprising enriched stromal and stem cells resulting in a second lipo-aspirate; and
d) concentrating the mesenchymal cell population of the second lipo-aspirate in a second centrifugation vessel in fluid communication with said first vessel via filter-centrifugation, wherein one or more filtering components or centrifuge tube strainers (CTS) of the second vessel are configured to allow flow-through of the ADSCs such that said ADSCs pellet below said CTS, wherein the second lipo-aspirate is centrifuged through said one or more filtering components or CTS, thereby resulting in a majority of the PIF with cell debris above the CTS which ADSCs are below the CTS, and wherein the PIF within the CTS is removed from the pelleted ADSCs.

2. The method of claim 1, wherein the PIF is saline, Ringer's solution or lactated Ringer's solution.

3. The method of claim 1, wherein the tubular device is a cannula having a diameter of between about 3 mm and 4 mm.

4. The method of claim 1, wherein the one or more first filters have a pore size of about 10 to 250 microns.

5. The method of claim 4, wherein the one or more first filters are composed of a material selected from the group consisting of glass fiber, polyester fiber, plastic fiber, metal fiber, composite cellulose and synthetic fiber, nylon mesh, polyester mesh, synthetic fabric, and combinations thereof.

6. The method of claim 1, wherein waste material is collected in one or more reservoirs in fluid communication with said vessels.

7. The method of claim 1, wherein the first vessel is configured to be deposited on a platform of an orbital shaker, and further comprising subjecting said first lipoaspirate to rotational speed of between about 50 to 250 rpm.

8. The method of claim 1, wherein said first vessel and second vessel is configured to be deposited in a centrifuge, and further comprising subjecting said first and second lipoaspirate to centrifugation at about 500 rpm to 1500 rpm.

9. The method of claim 8, wherein said first and second vessel are in fluid communication.

10. The method of claim 1, wherein said second one or more filters have a pore size of less than about 50 microns.

11. The method of claim 1, wherein said second one or more filters are composed of a material selected from the group consisting of glass fiber, polyester fiber, plastic fiber, metal fiber, composite cellulose and synthetic fiber, nylon mesh, polyester mesh, synthetic fabric, and combinations thereof.

12. The method of claim 1, wherein the second vessel is in fluid communication with an output reservoir or a sample chamber.

13. A method of treating a subject in need thereof with an enriched stem cell population from adipose tissue comprising:
a) injecting a physiological infiltration fluid (PIF) into a subject in one or more areas where fat is to be removed;
b) mechanically dissociating fatty tissue in the infiltrated area with a tubular device and removing the dissociated fatty tissue via aspiration;
c) collecting a first lipo-aspirate in a first vessel in fluid communication with said tubular device, wherein said first vessel comprises one or more first filters, and wherein said filters are configured to retain adipocyte globules and/or aggregates, thereby removing fat from infiltration fluid comprising an enriched mesenchymal cell population resulting in a second lipo-aspirate;
d) concentrating the mesenchymal cell population of the second lipo-aspirate in a second centrifugation vessel in fluid communication with said first vessel via filter-centrifugation, wherein one or more filtering components or centrifuge tube strainers (CTS) of the second vessel are configured to allow flow-through of the ADSCs such that said ADSCs pellet below said CTS, wherein the second lipo-aspirate is centrifuged through said one or more filtering components or CTS, thereby resulting in a majority of the PIF with cell debris above the CTS which ADSCs are below the CTS, and wherein the PIF within the CTS is removed from the pelleted ADSCs;
e) collecting the retained mesenchymal cell population in a sample reservoir in fluid communication with said second vessel; and
f) administering the mesenchymal cells in the sample reservoir to said subject.

14. The method of claim 13, wherein the isolated cells may be administered within about 1 to 3 hours of the initial injection of the PIF into said subject.

15. The method of claim 13, wherein the tubular device is a cannula having a diameter of between about 3 mm and 4 mm.

16. The method of claim 13, wherein the one or more first filters have a pore size of about 10 to 250 microns.

17. The method of claim 13, wherein the one or more first filters are composed of a material selected from the group consisting of glass fiber, polyester fiber, plastic fiber, metal fiber, composite cellulose and synthetic fiber, nylon mesh, polyester mesh, synthetic fabric, and combinations thereof.

18. The method of claim 13, wherein waste material is collected in one or more reservoirs in fluid communication with said vessels.

19. The method of claim 13, wherein the first vessel is configured to be deposited on a platform of an orbital shaker, and further comprising subjecting said first lipoaspirate to rotational speed of between about 50 to 250 rpm.

20. The method of claim 13, wherein said first vessel and second vessel is configured to be deposited in a centrifuge, and further comprising subjecting said first and second lipoaspirate to centrifugation at about 500 rpm to 1500 rpm.

21. The method of claim 13, wherein said second one or more filters have a pore size of less than about 50 microns.

22. The method of claim 13, wherein said second one or more filters are composed of a material selected from the group consisting of glass fiber, polyester fiber, plastic fiber, metal fiber, composite cellulose and synthetic fiber, nylon mesh, polyester mesh, synthetic fabric, and combinations thereof.

23. The method of claim 13, wherein the second vessel is in fluid communication with an output reservoir or a sample chamber.

24. An enriched fraction of adipocyte derived stem cells (ADSC) isolated by the method of claim 1, wherein in the absence of enzymatic dissociating agents the enriched fraction of cells possess increased levels of mRNA encoding collagen.

25. The enriched fraction of claim 24, wherein the enriched fraction of cells possess decreased levels of mRNA encoding MMP-1 (collagenase) when compared to ADSC obtained using enzymatic dissociating agents.

26. The enriched fraction of claim 24, wherein said ADSC cells express one or more cell surface markers selected from the group consisting of CD9 (tetraspan), CD10 (CALLA), CD13 (aminopeptidase), CD29 (β-1 integrin), CD44 (hyaluronate receptor or phagocytic glycoprotein-1), CD49d (α-4 integrin), CD49e (α-5 integrin), CD51 (α-V integrin), CD54 (ICAM-1), CD55 (DAF), CD59 (complement protectin), CD71 (transferrin receptor), CD73 (5′ nucleotidase), CD90 (Thy-1), CD105 (Endoglin), CD117 (c-Kit), CD146 (Muc18), CD166 (ALCAM), α-smooth muscle actin, Collagen type I, Collagen type II, HLA-ABC, Osteopontin, Osteonectin, and Vimentin.
